(19) United States
(12) Patent Application Publication
(10) Pub. No.: US 2026/0212951 A1
(43) Pub. Date: Jul. 23, 2026

(54) TARGET CONDITIONED SYNTHETIC PROTEIN BINDER GENERATION

(71) Applicant: NVIDIA Corporation, Santa Clara, CA (US)

(72) Inventors: Kieran Didi, Euskirchen (DE); Karsten Kreis, Vancouver (CA); Zuobai Zhang, Santa Clara, CA (US); Guoqing Zhou, Placentia, CA (US); Danny Reidenbach, San Jose, CA (US); Zhonglin Cao, San Jose, CA (US); Tomas Geffner, Santa Clara, CA (US); Christian Dallago, Munich (DE); Emine Kucukbenli, Melrose, MA (US); Arash Vahdat, San Mateo, CA (US)

(21) Appl. No.: 19/431,934

(22) Filed: Dec. 23, 2025

Related U.S. Application Data
(60) Provisional application No. 63/746,738, filed on Jan. 17, 2025.
(60) Provisional application No. 63/887,341, filed on Sep. 24, 2025.

(51) Int. Cl. G16B 15/30; G16B 15/20; G16B 40/20

(52) U.S. Cl. CPC G16B 15/30; G16B 15/20; G16B 40/20

(57) ABSTRACT

Designing binding proteins is a central challenge in computational biology. Modern artificial intelligence (AI)-based approaches fall into two classes: (1) generative methods which treat binder design as conditional generation, training on binder-target complex structures and producing new candidates for unseen targets; and (2) hallucination methods which use the confidence and alignment scores of structure predictors to assess interfaces and optimize binder sequences via gradient feedback. However, these current approaches do not unify the generative and hallucination methods. The present disclosure provides synthetic protein binder generation conditioned on a given target protein via a framework that unifies a flow-based base generative model with flexible inference-time optimization utilizing the generative prior, thereby combining the strengths of both approaches.

### CLAIM OF PRIORITY

This application claims the benefit of U.S. Provisional Application No. 63/746,738 (Attorney Docket No. NVIDP1434+/25-SC-0070US01) titled “EFFICIENT AND CONTROLLABLE PROTEIN BINDER DESIGN,” filed Jan. 17, 2025, and claims the benefit of U.S. Provisional Application No. 63/887,341 (Attorney Docket No. NVIDP1434++/25-SC-0070US02) titled “SCALING ATOMISTIC PROTEIN BINDER DESIGN WITH GENERATIVE PRETRAINING AND TEST-TIME COMPUTE,” filed Sep. 24, 2025, the entire contents of which are incorporated herein by reference.

### TECHNICAL FIELD

The present disclosure relates to protein structure synthesis.

### BACKGROUND

Designing binding proteins is a central challenge in computational biology. As protein interactions are mediated by structure, most binder design methods adopt a structure-centric view. Advances in machine learning for protein structure prediction and structure generation now enable increasingly accurate de novo binder design in silico. Modern artificial intelligence (AI)-based approaches fall into two classes: (1) generative methods which treat binder design as conditional generation, training on binder-target complex structures and producing new candidates for unseen targets; and (2) hallucination methods which use the confidence and alignment scores of structure predictors to assess interfaces and optimize binder sequences via gradient feedback.

This dichotomy stands in contrast to the large generation systems in language and image modeling, where a pretrained base model is combined with adaptive inference-time compute scaling and reasoning in a single framework. By analogy, current binder design methods resemble either pure training-time optimization (generative) or pure inference-time optimization without generative prior (hallucination), without allowing for unification of the generative model with the inference-time optimization.

There is thus a need for addressing these issues and/or other issues associated with the prior art. For example, there is a need to provide synthetic protein binder generation conditioned on a given target protein via a framework that unifies a flow-based base generative model with flexible inference-time optimization utilizing the generative prior, thereby combining the strengths of both approaches.

### SUMMARY

A method, computer readable medium, and system are disclosed for synthetic protein binder generation. In an embodiment, a flow-based generative model is trained to generate a synthetic protein binder design conditioned on a target protein structure, and the trained flow-based generative model is output. In another embodiment, a synthetic protein binder design conditioned on a target protein structure is generated, by a flow-based generative model, and the synthetic protein binder design is output.

### BRIEF DESCRIPTION OF THE DRAWINGS

FIG. 1A illustrates a flowchart of a method for training a flow-based generative model to generate synthetic protein binder designs, in accordance with an embodiment.

FIG. 1B illustrates a flowchart of a method for using a flow-based generative model to generate synthetic protein binder designs, in accordance with an embodiment.

FIG. 2 illustrates a system pipeline for training and inference operations corresponding to a flow-based generative model, in accordance with an embodiment.

FIG. 3 illustrates a flow of the system pipeline of FIG. 2, in accordance with an embodiment.

FIG. 4 illustrates exemplary synthetic protein binder designs, in accordance with an embodiment.

FIG. 5A illustrates inference and/or training logic, according to at least one embodiment.

FIG. 5B illustrates inference and/or training logic, according to at least one embodiment.

FIG. 6 illustrates training and deployment of a neural network, according to at least one embodiment.

FIG. 7 illustrates an example data center system, according to at least one embodiment.

### DETAILED DESCRIPTION

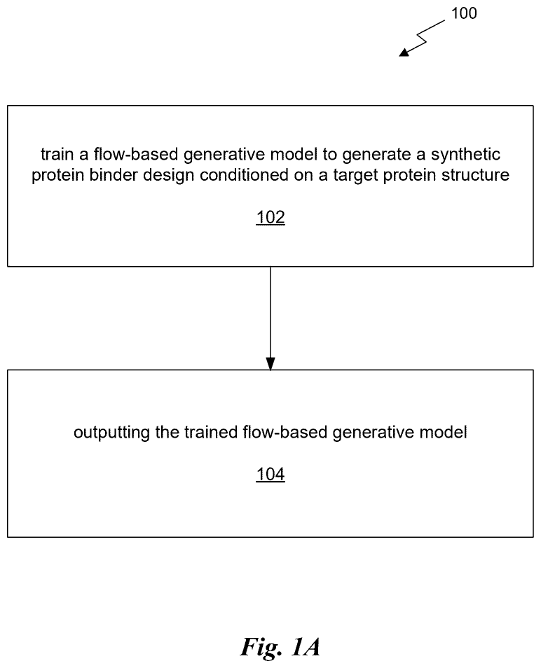

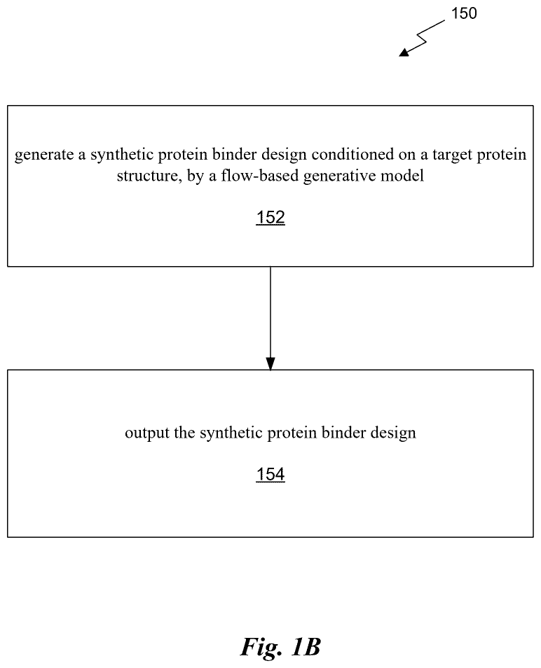

FIG. 1A illustrates a flowchart of a method 100 for training a flow-based generative model to generate synthetic protein binder designs, in accordance with an embodiment. The method 100 may be performed by a device, which may be comprised of a processing unit, a program, custom circuitry, or a combination thereof, in an embodiment. In another embodiment, a system comprised of a non-transitory memory storage comprising instructions, and one or more processors in communication with the memory, may execute the instructions to perform the method 100. In another embodiment, a non-transitory computer-readable media may store computer instructions which when executed by one or more processors of a device cause the device to perform the method 100.

In operation 102, a flow-based generative model is trained to generate a synthetic protein binder design conditioned on a target protein structure. With respect to the present description, the synthetic protein binder design is a computer generated design of a protein binder. The protein binder, also referred to as a binding protein, is a protein that binds to (e.g. couples to, interacts with, etc.) a target protein structure. The target protein structure can be a protein itself or a component thereof.

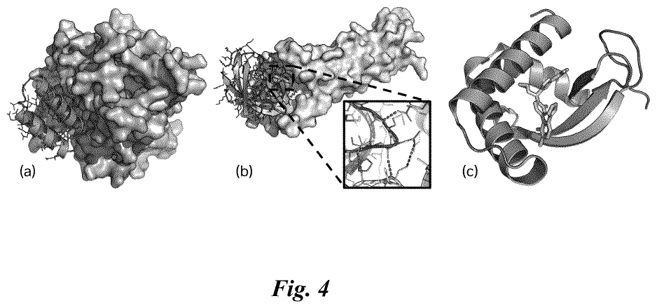

In an embodiment, the synthetic protein binder design may be generated such that it defines a realistic protein binder, whereby a physical protein formed (e.g. in a lab) in accordance with the synthetic protein binder design can be used in various applications, such as creating medicine for drug development, tissue engineering, regenerative medicine, biotechnology for research and industrial applications, material science for novel materials with unique properties, etc. FIG. 4 illustrates various exemplary (e.g. realistic) synthetic protein binder designs that may be generated by the flow-based generative model.

As mentioned, operation 102 includes training a flow-based generative model to generate synthetic protein binder designs conditioned on target protein structures. In other words, the flow-based generative model trained such that, given an input target protein structure, the flow-based generative model generates a design of a protein that, in the real world, will bind to a protein comprised of the target protein structure. In an embodiment, the flow-based generative model is trained in particular to generate a novel, fully atomistic protein binder predicted to interact with the target protein structure.

With respect to the present description, the flow-based generative model refers to a generative model that is configured to use flow-matching for generating synthetic protein binder designs. In an embodiment, the flow-based generative model may be pretrained prior to the training in operation 102. In an embodiment, the flow-based generative model may be pretrained on monomeric proteins, to learn a partially latent representation of proteins. In an embodiment, the flow-based generative model may be trained per operation 102 to only generate the synthetic protein binder design without generating a full target-binder complex.

In an embodiment, the flow-based generative model may be trained on pairs of protein binders and target proteins to learn their structural relationships. In an embodiment, the flow-based generative model may include a denoiser that is trained using each of the pairs of protein binders and target proteins. In an embodiment, the denoiser may be trained using one of the protein binder-target protein pairs by: adding noise to the protein binder (making a noisy protein binder), predicting, by the flow-based generative model, velocities relative to the noise, and optimizing the flow-based generative model based on the prediction. Further to this embodiment, the flow-based generative model may learn to denoise the noisy protein binder, such that at inference-time, the flow-based generative model can be initialize from random noise and, in iterative fashion, can generate a novel synthetic protein binder design. In an embodiment, during training the model may be conditioned on hotspots on the target protein structure, which guide the model to generate the protein binder design in proximity to the hotspots. In another embodiment, the model may be explicitly conditioned on other placement information (e.g. the binder center of mass), such that model will learn to denoise in a manner so the protein binder is placed according to this placement information. In yet another embodiment, global perturbation may be added in addition to the per-residue and per-latent-variable noise, which is essentially global noise added to the protein binder.

In an embodiment, the flow-based generative model may further be trained to enable fold conditioning when generating synthetic protein binder designs. In particular, the flow-based generative model may be trained to generate a synthetic protein binder design that is of one or more target (i.e. specified) fold classes. With respect to the present description, a fold class refers to a predefined category of protein tertiary structure topology. Thus, in this embodiment, the flow-based generative model may be trained to generate synthetic protein binder designs conditioned on both target protein structures and target fold class(es).

In an embodiment, the method 100 may further include, in addition to operation 102, performing reward-based fine-tuning of the trained flow-based generative model. In an embodiment, the reward-based fine-tuning may be performed by: calculating a reward based on predefined structural metrics that are favorable for successful synthetic protein binder design, and updating parameters of the trained flow-based generative model based on the reward.

In operation 104, the trained flow-based generative model is output. In an embodiment, the trained flow-based generative model is output to an execution environment (e.g. computer system, computing platform, etc.) for performing inference-time operations. The inference-time operations may include generating a new (e.g. novel) synthetic protein binder design conditioned on an input target protein structure. An embodiment of the inference-time use of the trained flow-based generative model is described below with reference to the method 150 of FIG. 1B.

In an embodiment, the trained flow-based generative model may be accessible to one or more applications, where the application operates to input a target protein structure to the flow-based generative model to cause the model to generate a synthetic protein binder design for the target protein structure. In this embodiment, the generated synthetic protein binder design may returned to the application for use in one or more downstream tasks, such as for use in generating the physical protein or physical protein-target complex (e.g. in a lab).

FIG. 1B illustrates a flowchart of a method 150 for using a flow-based generative model to generate synthetic protein binder designs, in accordance with an embodiment. The method 150 may be performed by a device, which may be comprised of a processing unit, a program, custom circuitry, or a combination thereof, in an embodiment. In another embodiment, a system comprised of a non-transitory memory storage comprising instructions, and one or more processors in communication with the memory, may execute the instructions to perform the method 150. In another embodiment, a non-transitory computer-readable media may store computer instructions which when executed by one or more processors of a device cause the device to perform the method 150.

In an embodiment, the flow-based generative model used per the method 150 may be pretrained in accordance with the method 100 of FIG. 1A. Thus, the definitions and descriptions provided above may equally apply to the present embodiment.

In operation 152, a synthetic protein binder design conditioned on a target protein structure is generated, by a flow-based generative model. In an embodiment, the synthetic protein binder design may be a novel, fully atomistic protein binder predicted to interact with the target protein structure. In an embodiment, the target protein structure may be input to the flow-based generative model to cause the model to generate the synthetic protein binder design. In an embodiment, the flow-based generative model may generate only the synthetic protein binder design without generating a full target-binder complex.

In an embodiment, the synthetic protein binder design generated by the flow-based generative model may be further conditioned on one or more target fold classes. Accordingly, the synthetic protein binder design generated by the flow-based generative model may be of the target fold class(es). In an embodiment, the synthetic protein binder design may be generated from noise, including for example a noisy input to the flow-based generative model.

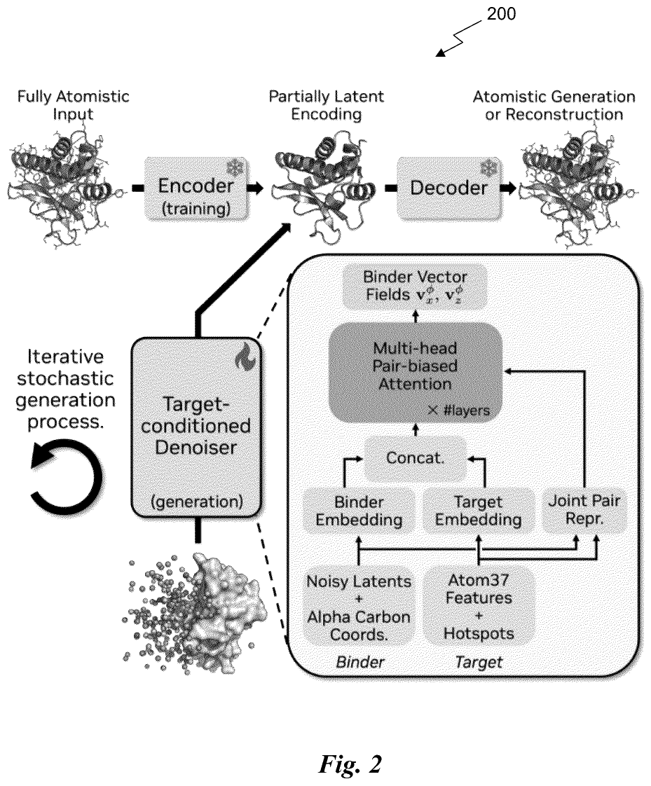

In an embodiment, operation 152 may include generating a predefined number of synthetic protein binder designs conditioned on the target protein structure, by the flow-based generative model. In an embodiment, the predefined number may be greater than one. In this embodiment, the method 150 may further include performing an inference-time optimization on the synthetic protein binder designs to select one or more of the synthetic protein binder designs for output. The inference-time optimization may be performed in accordance with a preconfigured optimization function, such as for example best-of-N sampling, beam searching, Feynman-Kac steering, Monte Carlo tree searching, each of which is described in more detail below with reference to FIG. 2. The inference-time optimization may involve an evaluation of generated synthetic protein binder designs using a folding model, for example by evaluating with the folding model's confidence scores. In another embodiment, the generated synthetic protein binder designs may be evaluated without a folding model, but instead by using for example energy functions that measure binding to the inference.

In an embodiment, the synthetic protein binder design may be generated by the flow-based generative model via an inference-time optimization that includes a rollout process. The rollout process may include, for each of a plurality of steps of the rollout process and for each of one or more denoising trajectories, generating a plurality of candidate states for the denoising trajectory, scoring each of the plurality of candidate states, and selecting one or more of the plurality of candidate states to update the denoising trajectory, based on the scores. A final synthetic protein binder design resulting from the inference-time optimization may be the synthetic protein binder design generated by the flow-based generative model.

In operation 154, the synthetic protein binder design is output. In an embodiment, the synthetic protein binder design is output to a memory. In an embodiment, the synthetic protein binder design may be output for use in generating a physical protein. In an embodiment, the method 150 may further include generating the physical protein from the synthetic protein binder design (e.g. using an automated manufacturing system). In an embodiment, the synthetic protein binder design may be output to a downstream application for use in one or more downstream tasks, such as for use in generating the physical protein or physical protein-target complex (e.g. in a lab). In an embodiment, the method 150 may further include the downstream application using the synthetic protein binder design and/or the physical protein as mentioned above.

Further embodiments will now be provided in the description of the subsequent figures. It should be noted that the embodiments disclosed herein with reference to the method 100 of FIG. 1A and/or the method 150 of FIG. 1B may apply to and/or be used in combination with any of the embodiments of the remaining figures below.

FIG. 2 illustrates a system pipeline 200 for training and inference operations corresponding to a flow-based generative model, in accordance with an embodiment. The system pipeline 200 may be implemented to carry out the method 100 of FIG. 1A and/or the method 150 of FIG. 1B. Thus, the definitions and descriptions provided above may equally apply to the present embodiment.

The system pipeline 200 is built on a framework that is configured to use flow-matching for generation, which models a probability path pₜ(xₜ) transforming tractable noise pₜ₌₀ into data pₜ₌₁ via an ordinary differential equation (ODE) dxₜ=v^{θ}(xₜ, t)dt defined by a learnable vector field v^{θ}. Training uses conditional flow matching (CFM), where conditional paths pₜ(xₜ|x₁) make the target vector field uₜ(xₜ|x₁) tractable for simple p₀. The network is trained by regressing v^{θ} against uₜ(xₜ|x₁), yielding in expectation the same gradients as regression on the intractable marginal field uₜ(xₜ). The framework adopts the rectified flow formulation with a linear interpolant xₜ=tx₁+(1−t)x₀ and target x₁−x₀ to model fully atomistic proteins using a partially latent flow matching framework. Specifically, it performs joint flow matching over residues' alpha carbon coordinates x^{c}^{_{α}} and per-residue continuous latent variables z that encode amino acid identities s and the residues' remaining atom coordinates x^{-c}^{_{α}}. This partially latent representation emerges from a variational autoencoder (VAE) framework with encoder ε(x^{c}^{_{α}}, x^{-c}^{_{α}}) and decoder D(x^{c}^{_{α}}, z), corresponding to approximate posterior and conditional likelihood in the VAE formalism, respectively. In an embodiment, the decoder outputs atom coordinates in an Atom37 protein representation. In an embodiment, in addition to these atom coordinates, the decoder also outputs the amino acid identities of each residue.

As mentioned, the system pipeline 200 is built on top of the aforementioned framework, which offers state-of-the-art accurate fully atomistic protein generation capabilities, necessary to produce precise atomistic binder-target interfaces, and which is scalable and efficient, relying on fast transformer networks without slow triangular multiplicative or attention layers which is critical as binder design often involves in-silico generation of many candidates, especially when scaling compute during inference as described in more detail below. The system pipeline 200 introduces the following adaptations to extend the aforementioned framework to binder design.

Target- and Hotspot-Conditioning. The system pipeline 200 generates partially latent representations of binder proteins only. Thus, only the partially latent flow matching model conditions on the target, while the autoencoder simply encodes and decodes monomeric binders. To represent the target, the Atom37 scheme may be used in an embodiment, i.e. each residue is assigned up to 37 three-dimensional atomic coordinates, determined by its amino acid type. These residue-wise Atom37 features are combined with amino acid identity features and binary hotspot tokens that mark interface residues near which the binder should be generated. During training, hotspots are extracted from interface residues of binder-target training pairs. In an embodiment, interface information may be provided for a subset of training samples by either 1) initializing the noise cloud approximately at the center of origin or 2) providing an interface token in a target area, where the noise cloud is still initialized at the origin but the token allows the network to learn that it should roughly put the binder in that target area. Further to this embodiment, remaining training samples may not have interface information and therefore may require the model to learn that information itself. In this case, the noise cloud should still not be initialized inside the center, but within a preconfigured distance away from the target in a random direction.

During inference, hotspots may be known, or otherwise preprocessing can be used for hotspot identification. The resulting target-conditioning features, denoted as c^{target}, are linearly embedded and concatenated in the token dimension to the sequence of alpha carbon coordinates x^{c}^{_{α}} and latent variables z that encode the binder. This extended sequence of noisy binder embeddings and clean target embeddings is then jointly processed by the transformer denoiser network of the partially latent flow model, which applies pair-biased attention. Pair representations are formed jointly over the extended sequence representation of binder and target. The latent target conditioning mechanism is illustrated in FIG. 2.

### Architectural Details

The system pipeline 200 operates in a conditional setting: it receives a target protein structure as input and generates a novel, fully atomistic protein binder predicted to interact with that target. As mentioned, the VAE architecture is preliminarily trained on monomeric proteins, to learn a partially latent representation of proteins. The partially latent flow matching model is then trained to generate a binder protein conditioned on a given target. This approach does not generate the full target-binder complex, but instead the target protein serves exclusively as conditioning information. While the neural network architecture for the system pipeline 200 processes the binder and target jointly to learn their structural relationship, the generative output of our flow matching model is solely the binder.

The model's denoiser is built upon a transformer architecture that uses pair-biased attention mechanisms. The denoiser network is designed to process a target protein (coordinates and sequence) alongside a potentially noisy, partially latent representation of a binder (α-carbon coordinates and latent variables). These inputs are jointly featurized into two tensors: a sequence representation with shape [L_{binder}+L_{target}, c_{seq}] and a pair representation with shape [L_{binder}+L_{target}, L_{binder}+L_{target}, cₚₐᵢᵣ]. Intuitively, the sequence representation encodes residue-level features, while the pair representation captures features between pairs of residues (e.g., inter-atomic distances). The transformer blocks iteratively update the sequence representation, with the pair representation used to bias the attention logits through linear layers. The pair representation remains static throughout the network. By omitting triangular update layers, the model is highly efficient, a property that is particularly advantageous for applying inference-time scaling techniques. In an exemplary embodiment, c_{seq}=768, cₚₐᵢᵣ=256, and 14 transformer layers are used. This leads to a denoiser architecture of ~160M parameters.

Although the denoiser network processes a joint representation of the target and binder, its generative output is the velocity field for the binder.

Conditioning on diffusion times and CAT embeddings. In the present description, CAT stands for a hierarchy of three labels, “Class”, “Architecture”, and “Topology”, that describe the protein fold at different levels of complexity. The denoiser is conditioned on several inputs beyond the protein structures themselves. First, it receives the diffusion times, tₓ and t_{z}, which correspond to the noise levels of the binder's α-carbon coordinates and latent variables, respectively. These time values are integrated into the network's transformer blocks using adaptive layer normalization and output scaling techniques. Additionally, the model is conditioned on the structural class of the target and binder proteins using their CAT labels. Each CAT label is mapped to a learnable embedding, which is then incorporated into the network using the same adaptive normalization and output scaling layers.

### Features Used as Input to the Denoiser

### Initial Sequence Representation.

The initial sequence representation is a concatenation of two independently constructed tensors: one for the binder, with shape [L_{binder}, c_{seq}], and one for the target, with shape [L_{target}, c_{seq}]. For the binder, the per-residue feature vector is derived from three sources: (i) the raw coordinates of the noisy α-carbons, (ii) the raw values of the noisy latent variables, and (iii) a Fourier embedding of the residue index. These features are concatenated and projected through a linear layer to produce the initial binder feature tensor. For the target, the feature vector is constructed from five sources: (i) a one-hot representation of the amino acid sequence, (ii) the full-atom structure (Atom37 representation), (iii) a binary indicator for hotspot residues, (iv) backbone torsion angles, binned into 20 bins between −π and π, and (v) side chain angles, also binned into 20 bins between −π and π. These are similarly concatenated and passed through a linear layer to yield the target's feature tensor. Finally, the binder and target sequence representations are concatenated along the length dimension to form a single input tensor of shape [L_{binder}+L_{target}, c_{seq}].

### Initial Pair Representation.

A similar modular construction is used for the initial pair representation, which has a final shape of [L_{binder}+L_{target}, L_{binder}+L_{target}, cₚₐᵢᵣ]. This tensor is assembled from three distinct sub-tensors: one describing interactions within the binder, one for interactions within the target, and one for the cross-interactions between the binder and target.

The binder-binder pair representation, with shape [L_{binder}, L_{binder}, cₚₐᵢᵣ], has features that include (i) the relative sequence separation, one-hot encoded and limited to ±64 residues, and (ii) the pairwise distances between the noisy α-carbon coordinates, bucketized into 1 Å bins between 1 Å and 30 Å. These features are concatenated and projected through a linear layer.

The target-target pair representation, with shape [L_{target}, L_{target}, cₚₐᵢᵣ], is derived from features that describe intra-target residue pairs. These include: (i) the relative sequence separation, one-hot encoded and limited to ±64 residues, (ii) the pairwise distances between backbone atoms, bucketized into 1 Å bins between 1 Å and 30 Å, (iii) a binary chain index (0 if residues are from the same chain, 1 if different), and (iv) a hotspot pair variable (0 if neither residue is a hotspot, 1 if one is, and 2 if both are). These features are concatenated and fed into a linear layer for the target pair representation.

The cross-pair representation, describing binder-target interactions with shape [L_{binder}, L_{target}, cₚₐᵢᵣ], is built from features capturing inter-protein residue relationships. (Note: these features are analogous to the target-target features but are computed between binder-target residue pairs). They include: (i) the pairwise distances between the binder's noisy α-carbon coordinates and the target's backbone atoms, bucketized into 1 Å bins between 1 Å and 30 Å, (ii) a binary chain index, and (iii) a hotspot pair variable (note that there are no hotspots in the binder, therefore in this case this variable can only be 0 or 1). These features are concatenated and fed into a linear layer for the cross-pair representation.

Finally, these three tensors are assembled into the full initial pair representation that is passed to the network. This feature construction scheme is inherently compatible with multi-chain targets, where such targets are simply treated as a single entity, where the chain index feature naturally distinguishes between residues belonging to different chains.

In embodiments, the target representations can increase efficiency and incorporate conformational flexibility. Embodiments can circumvent the need for rigid targets by 1) leveraging new paired datasets that allow training on targets in their bound and unbound form, thereby giving the model the ability to learn about flexibility and include it in the generation process and 2) changing the target representation so that the model does not rely on a fixed bound representation throughout the generation trajectory. This can be achieved either via a) direct conditioning of a simple target structure encoding via adaptive layer norms, similar to how other features like fold-class conditioning are implemented, b) encoding the target structure into a fixed-size representation, for example via an autoencoder whose representation then serves as a basis for cross-attention of the binder to this representation, or c) integrating specific attention mechanisms between interface residues.

Objective with Translation Noise. Using the latent target conditioning architecture described above, the partially latent flow model's objective is defined per Equation 1.

$$\begin{matrix}min {𝔼}_{{t}_{x} , {t}_{z} ( x , {c}^{target} ) ∼ {p}^{data} , {x}_{0}^{{C}_{α}} ∼ {p}_{0}^{{C}_{α}} , {z}_{0} ∼ {p}_{0}^{z} , d ∼ {p}_{0}^{d}} \\ ∅\end{matrix} [ {‖ {v}_{z}^{∅} ( {x}_{{t}_{x}}^{{C}_{α}} , {z}_{{t}_{z}} , {c}^{target} , {t}_{x} , {t}_{z} ) - ( ε ( x ) - {z}_{0} ) ‖}^{2} + {‖ {v}_{x}^{∅} ( {x}_{{t}_{x}}^{{C}_{α}} , {z}_{{t}_{z}} , {c}^{target} , {t}_{x} , {t}_{z} ) - ( {x}^{{C}_{α}} - [ {x}_{0}^{{C}_{α}} + d 1 ] ) ‖}^{2} ]$$

where x and c_{target} denote binder and target drawn from the training data distribution p^{data}, (x) is the monomer encoder applied to the binder x, and tₓ and t_{z} are the interpolation times of alpha carbon coordinates and latents, respectively. Moreover,

$${x}_{{t}_{x}}^{{C}_{α}} = {t}_{x} {x}^{{C}_{α}} + ( 1 - {t}_{x} ) ( {x}_{0}^{{C}_{α}} + d 1 ) , {z}_{{t}_{z}} = {t}_{z} ε ( x ) + ( 1 - {t}_{z} ) {z}_{0} , {p}_{0}^{{C}_{α}} = N ( {x}_{0}^{{C}_{α}} \vert 0 , I ) , and {p}_{0}^{z} = N ( {z}_{0} \vert 0 , I ) .$$

The binder's alpha carbon coordinates are perturbed with a scalar random global translation

$$d ∼ {p}_{0}^{d} = N ( d \vert 0 , {c}_{d}^{2} )$$

when applying the interpolant (c_{d}=0.2 nm, in an embodiment). This explicit translation noise forces the model to reason over global positioning of the protein. This is irrelevant in monomer generation, but critical in binder design, when the binder needs to be accurately positioned at the interface. This additional translation noise forces the model to refine positioning throughout generation.

Stagewise Training. In an embodiment, the system pipeline 200 may employ multi-stage training. First, the autoencoder is trained on monomers. In an embodiment, the autoencoder may be first trained on monomers from the AlphaFold Database (AFDB), then fine-tuned on monomers from the Protein Databank (PDB). Next, the partially latent flow-matching model is pretrained on encoded cluster representatives of the AFDB, enabling it to acquire general protein structure generation capabilities. Only afterwards is the model trained on binder target pairs.

Inference-Time Optimization. Prior hallucination methods can be seen as pure inference-time optimization without use of a generative model. To combine the flow-based generative model described above with interference-time optimization, test-time scaling methods are employed. In general, the partially latent flow model is sampled stochastically with reduced noise injection. This sampling may be performed in accordance with any of the following sampling techniques, by way of example.

Best-of-N Sampling is the simplest version of test-time compute scaling: Given an increasing compute budget, the number of generated samples N is grown and rewards computed using a preconfigured reward function (folding model), and all binders with at least a minimum reward (or meeting a pre-defined success criteria) are selected.

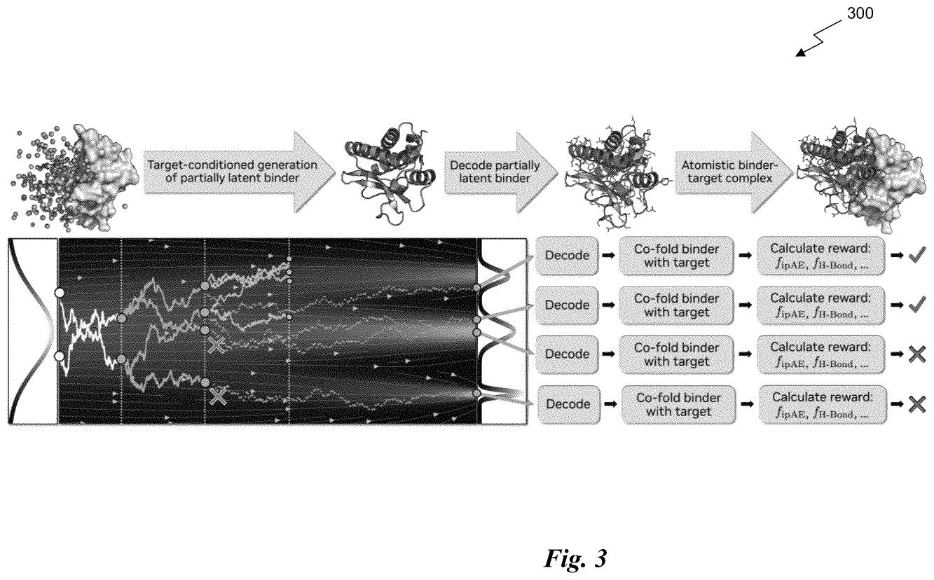

Beam Search (visualized in FIG. 3) includes maintaining a set of N (beam width) denoising trajectories

$${B}_{{t}_{x} , {t}_{z}} = {\{ ( {x}_{{t}_{x}}^{{C}_{α}} , {z}_{{t}_{z}} ) \}}_{i = 1}^{i = N} ,$$

the “beam”. From each beam element i, L (branching factor) new stochastic denoising trajectories are initiated that are then run for K denoising steps to obtain a total of N×L new candidate states

$${C}_{{t}_{w} + Δ {t}_{x}^{K} , {t}_{z} + Δ {t}_{z}^{K}} = {\{ {( {x}_{{t}_{x} + Δ {t}_{x}^{K}}^{{C}_{α}} , {z}_{{t}_{z} + Δ {t}_{z}^{K}} )}_{i} \}}_{i = 1}^{i = NL} .$$

All candidates i are now stochastically simulated towards clean partially latent states, decoded, the resulting sequences folded, and the candidates' rewards

$$R ( {( {x}_{{t}_{x} + Δ {t}_{x}^{K}}^{{C}_{α}} , {z}_{{t}_{z} + Δ {t}_{z}^{K}} )}_{i} )$$

calculated. The top-N candidates can then be selected to form the updated beam after K steps, which can be formalized per Equation 2.

$${B}_{{t}_{x} + Δ {t}_{x}^{K} , {t}_{z} + Δ {t}_{z}^{K}} = \begin{matrix}arg max \\ τ ⊆ C , \vert τ \vert = N\end{matrix} {\sum }_{i ∈ τ} R ( {( {x}_{{t}_{x} + Δ {t}_{x}^{K}}^{{C}_{α}} , {z}_{{t}_{z} + Δ {t}_{z}^{K}} )}_{i} ) \qquad Equation 2$$

This procedure is continued until fully denoised samples are obtained. In contrast to prior work all candidate states are iteratively rolled out. While this leads to stochastic rewards, the resulting samples on which decoder and structure predictor operate are clean, which is necessary because the structure prediction-based rewards are only reliable on realistic sequences. Due to the efficiency of the model, rolling out full generation trajectories is computationally inexpensive, and this search is performed only every K steps.

Feynman-Kac Steering (FKS) is related to beam search, but instead of top-N it uses importance sampling to sample the tilted distribution p^{Ø}(x^{c}^{_{α}}, z)exp{βR(x^{c}^{_{α}}, z)}, where p^{Ø} denotes the model distribution and β is an inverse temperature scaling. Specifically, N new states are sub-sampled from the N×L candidates

$${C}_{{t}_{x} + Δ {t}_{x}^{K} , {t}_{z} + Δ {t}_{z}^{K}}$$

with probability per Equation 3.

$$p ( ( {x}_{{t}_{x} + Δ {t}_{x}^{K}}^{{C}_{α}} , {z}_{{t}_{z} + Δ {t}_{z}^{K}} ) i ) ∝ exp \{ β R ( {( {x}_{{t}_{x} + Δ {t}_{x}^{K}}^{{C}_{α}} , {z}_{{t}_{z} + Δ {t}_{z}^{K}} )}_{i} ) \} \qquad Equation 3$$

Monte Carlo Tree Search (MCTS) treats the iterative generation process of flow and diffusion models as a tree, where different paths in the tree correspond to different stochastic denoising trajectories, and MCTS then searches within that tree. The method explores many possible denoising paths and then chooses the best next state along the tree. It is critical to balance exploration and exploitation when traversing the denoising tree (via parameter C in Equation 4) and child states

$${( {x}_{{t}_{x} + Δ {t}_{x}^{K}}^{{C}_{α}} , {z}_{{t}_{z} + Δ {t}_{z}^{K}} )}_{i}$$

are chosen according to the index selection criterion as shown in Equation 4.

$$i = \begin{matrix}arg max \\ i\end{matrix} \frac{R ( {( {x}_{{t}_{x} + Δ {t}_{x}^{K}}^{{C}_{α}} , {z}_{{t}_{z} + Δ {t}_{z}^{K}} )}_{i} )}{{\underbrace{V ( {( {x}_{{t}_{x} + Δ {t}_{x}^{K}}^{{C}_{α}} , {z}_{{t}_{z} + Δ {t}_{z}^{K}} )}_{i} )}}_{exploitation}} + C {\underbrace{\sqrt{\frac{ln ( V ( {( {x}_{{t}_{x}}^{{C}_{α}} , {z}_{{t}_{z}} )}_{i} ) )}{V ( {( {x}_{{t}_{x} + Δ {t}_{x}^{K}}^{{C}_{α}} , {z}_{{t}_{z} + Δ {t}_{z}^{K}} )}_{i} )}}}}_{exploitation} \qquad Equation 4$$

where

$${( {x}_{{t}_{x}}^{{C}_{α}} , {z}_{{t}_{z}} )}_{i}$$

denotes the parent of

$${( {x}_{{t}_{x} + Δ {t}_{x}^{K}}^{{C}_{α}} , {z}_{{t}_{z} + Δ {t}_{x}^{K}} )}_{i}$$

and V (·) counts how often a (noisy) partially latent state has already been visited during previous searches.

More specifically, each iteration of MCTS consists of four steps: selection, where the tree is traversed with a strategy to balance exploration and exploitation; expansion, where a new node for an unexplored action is added; simulation, where a rollout estimates the expected reward from that state; and backpropagation, where the results are propagated back up the tree to update visit counts and reward estimates. Repeating this process focuses the search on promising actions, making MCTS effective for complex tasks.

For effectiveness with flow-matching, selection and expansion are merged into a single stochastic decision at each node. At each step, with probability ϵ, a new child is expanded by running K denoising steps from the current node; with probability 1−ϵ, the child with the highest expected reward is selected. If the current node has no children or the next step reaches tₓ=t_{z}=1, a new child is always expanded. Once the current node reaches (tₓ, t_{z})=1, it will be decoded into a structure-sequence pair (x, s), and the reward is computed. This reward is then backpropagated to all ancestor nodes to update visit counts and expected rewards. Fully denoised states meeting the success criteria are added to a success set S, similar to beam search.

Generate and Hallucinate (G&H). The above approaches integrate search directly into the model's generative denoising process. A simpler approach can also be taken to combine generative with hallucination methods: initialize a binder candidate with the generative model, and then refine its sequence through an established hallucination method, such as one that uses several stages of optimization that partially rely on ad-hoc sequence relaxations to enable backpropagation through discrete sequences (in contrast to our Best-of-N, Beam Search, FKS, and MCTS variants, which are all principled and stable search algorithms).

### Filtering

In an embodiment, synthetic protein binder designs generated by the model may be filtered prior to inference-time optimization. This can preliminarily remove samples from consideration that will not be successful in the evaluation pipeline. In an embodiment, the filtering may be performed first by simple heuristics (e.g. protruding loops, radius of gyration, etc.) as well as log-likelihoods of the generated structure and sequence (e.g. based on external oracles as well as the model itself).

### Machine Learning

Deep neural networks (DNNs), including deep learning models, developed on processors have been used for diverse use cases, from self-driving cars to faster drug development, from automatic image captioning in online image databases to smart real-time language translation in video chat applications. Deep learning is a technique that models the neural learning process of the human brain, continually learning, continually getting smarter, and delivering more accurate results more quickly over time. A child is initially taught by an adult to correctly identify and classify various shapes, eventually being able to identify shapes without any coaching. Similarly, a deep learning or neural learning system needs to be trained in object recognition and classification for it get smarter and more efficient at identifying basic objects, occluded objects, etc., while also assigning context to objects.

At the simplest level, neurons in the human brain look at various inputs that are received, importance levels are assigned to each of these inputs, and output is passed on to other neurons to act upon. An artificial neuron or perceptron is the most basic model of a neural network. In one example, a perceptron may receive one or more inputs that represent various features of an object that the perceptron is being trained to recognize and classify, and each of these features is assigned a certain weight based on the importance of that feature in defining the shape of an object.

A deep neural network (DNN) model includes multiple layers of many connected nodes (e.g., perceptrons, Boltzmann machines, radial basis functions, convolutional layers, etc.) that can be trained with enormous amounts of input data to quickly solve complex problems with high accuracy. In one example, a first layer of the DNN model breaks down an input image of an automobile into various sections and looks for basic patterns such as lines and angles. The second layer assembles the lines to look for higher level patterns such as wheels, windshields, and mirrors. The next layer identifies the type of vehicle, and the final few layers generate a label for the input image, identifying the model of a specific automobile brand.

Once the DNN is trained, the DNN can be deployed and used to identify and classify objects or patterns in a process known as inference. Examples of inference (the process through which a DNN extracts useful information from a given input) include identifying handwritten numbers on checks deposited into ATM machines, identifying images of friends in photos, delivering movie recommendations to over fifty million users, identifying and classifying different types of automobiles, pedestrians, and road hazards in driverless cars, or translating human speech in real-time.

During training, data flows through the DNN in a forward propagation phase until a prediction is produced that indicates a label corresponding to the input. If the neural network does not correctly label the input, then errors between the correct label and the predicted label are analyzed, and the weights are adjusted for each feature during a backward propagation phase until the DNN correctly labels the input and other inputs in a training dataset. Training complex neural networks requires massive amounts of parallel computing performance, including floating-point multiplications and additions. Inferencing is less compute-intensive than training, being a latency-sensitive process where a trained neural network is applied to new inputs it has not seen before to classify images, translate speech, and generally infer new information.

### Inference and Training Logic

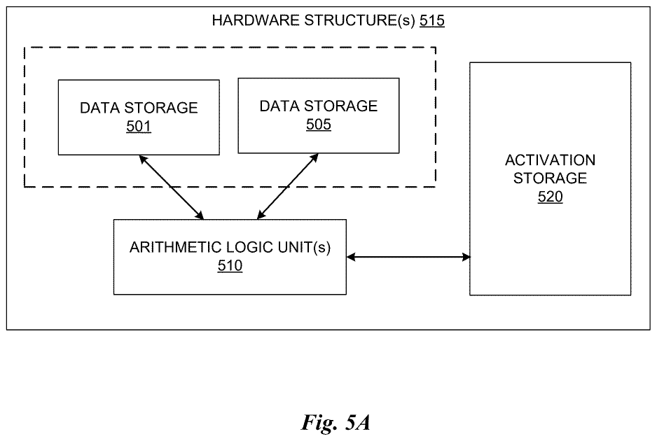

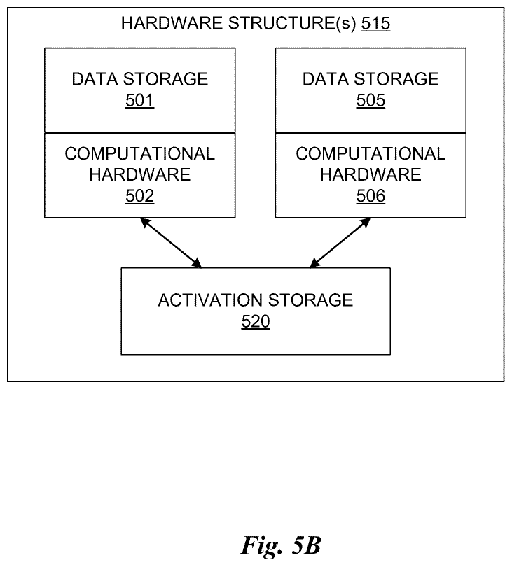

As noted above, a deep learning or neural learning system needs to be trained to generate inferences from input data. Details regarding inference and/or training logic 515 for a deep learning or neural learning system are provided below in conjunction with FIGS. 5A and/or 5B.

In at least one embodiment, inference and/or training logic 515 may include, without limitation, a data storage 501 to store forward and/or output weight and/or input/output data corresponding to neurons or layers of a neural network trained and/or used for inferencing in aspects of one or more embodiments. In at least one embodiment data storage 501 stores weight parameters and/or input/output data of each layer of a neural network trained or used in conjunction with one or more embodiments during forward propagation of input/output data and/or weight parameters during training and/or inferencing using aspects of one or more embodiments. In at least one embodiment, any portion of data storage 501 may be included with other on-chip or off-chip data storage, including a processor's L1, L2, or L3 cache or system memory.

In at least one embodiment, any portion of data storage 501 may be internal or external to one or more processors or other hardware logic devices or circuits. In at least one embodiment, data storage 501 may be cache memory, dynamic randomly addressable memory (“DRAM”), static randomly addressable memory (“SRAM”), non-volatile memory (e.g., Flash memory), or other storage. In at least one embodiment, choice of whether data storage 501 is internal or external to a processor, for example, or comprised of DRAM, SRAM, Flash or some other storage type may depend on available storage on-chip versus off-chip, latency requirements of training and/or inferencing functions being performed, batch size of data used in inferencing and/or training of a neural network, or some combination of these factors.

In at least one embodiment, inference and/or training logic 515 may include, without limitation, a data storage 505 to store backward and/or output weight and/or input/output data corresponding to neurons or layers of a neural network trained and/or used for inferencing in aspects of one or more embodiments. In at least one embodiment, data storage 505 stores weight parameters and/or input/output data of each layer of a neural network trained or used in conjunction with one or more embodiments during backward propagation of input/output data and/or weight parameters during training and/or inferencing using aspects of one or more embodiments. In at least one embodiment, any portion of data storage 505 may be included with other on-chip or off-chip data storage, including a processor's L1, L2, or L3 cache or system memory. In at least one embodiment, any portion of data storage 505 may be internal or external to on one or more processors or other hardware logic devices or circuits. In at least one embodiment, data storage 505 may be cache memory, DRAM, SRAM, non-volatile memory (e.g., Flash memory), or other storage. In at least one embodiment, choice of whether data storage 505 is internal or external to a processor, for example, or comprised of DRAM, SRAM, Flash or some other storage type may depend on available storage on-chip versus off-chip, latency requirements of training and/or inferencing functions being performed, batch size of data used in inferencing and/or training of a neural network, or some combination of these factors.

In at least one embodiment, data storage 501 and data storage 505 may be separate storage structures. In at least one embodiment, data storage 501 and data storage 505 may be same storage structure. In at least one embodiment, data storage 501 and data storage 505 may be partially same storage structure and partially separate storage structures. In at least one embodiment, any portion of data storage 501 and data storage 505 may be included with other on-chip or off-chip data storage, including a processor's L1, L2, or L3 cache or system memory.

In at least one embodiment, inference and/or training logic 515 may include, without limitation, one or more arithmetic logic unit(s) (“ALU(s)”) 510 to perform logical and/or mathematical operations based, at least in part on, or indicated by, training and/or inference code, result of which may result in activations (e.g., output values from layers or neurons within a neural network) stored in an activation storage 520 that are functions of input/output and/or weight parameter data stored in data storage 501 and/or data storage 505. In at least one embodiment, activations stored in activation storage 520 are generated according to linear algebraic and or matrix-based mathematics performed by ALU(s) 510 in response to performing instructions or other code, wherein weight values stored in data storage 505 and/or data 501 are used as operands along with other values, such as bias values, gradient information, momentum values, or other parameters or hyperparameters, any or all of which may be stored in data storage 505 or data storage 501 or another storage on or off-chip. In at least one embodiment, ALU(s) 510 are included within one or more processors or other hardware logic devices or circuits, whereas in another embodiment, ALU(s) 510 may be external to a processor or other hardware logic device or circuit that uses them (e.g., a co-processor). In at least one embodiment, ALUs 510 may be included within a processor's execution units or otherwise within a bank of ALUs accessible by a processor's execution units either within same processor or distributed between different processors of different types (e.g., central processing units, graphics processing units, fixed function units, etc.). In at least one embodiment, data storage 501, data storage 505, and activation storage 520 may be on same processor or other hardware logic device or circuit, whereas in another embodiment, they may be in different processors or other hardware logic devices or circuits, or some combination of same and different processors or other hardware logic devices or circuits. In at least one embodiment, any portion of activation storage 520 may be included with other on-chip or off-chip data storage, including a processor's L1, L2, or L3 cache or system memory. Furthermore, inferencing and/or training code may be stored with other code accessible to a processor or other hardware logic or circuit and fetched and/or processed using a processor's fetch, decode, scheduling, execution, retirement and/or other logical circuits.

In at least one embodiment, activation storage 520 may be cache memory, DRAM, SRAM, non-volatile memory (e.g., Flash memory), or other storage. In at least one embodiment, activation storage 520 may be completely or partially within or external to one or more processors or other logical circuits. In at least one embodiment, choice of whether activation storage 520 is internal or external to a processor, for example, or comprised of DRAM, SRAM, Flash or some other storage type may depend on available storage on-chip versus off-chip, latency requirements of training and/or inferencing functions being performed, batch size of data used in inferencing and/or training of a neural network, or some combination of these factors. In at least one embodiment, inference and/or training logic 515 illustrated in FIG. 5A may be used in conjunction with an application-specific integrated circuit (“ASIC”), such as Tensorflow® Processing Unit from Google, an inference processing unit (IPU) from Graphcore™, or a Nervana® (e.g., “Lake Crest”) processor from Intel Corp. In at least one embodiment, inference and/or training logic 515 illustrated in FIG. 5A may be used in conjunction with central processing unit (“CPU”) hardware, graphics processing unit (“GPU”) hardware or other hardware, such as field programmable gate arrays (“FPGAs”).

FIG. 5B illustrates inference and/or training logic 515, according to at least one embodiment. In at least one embodiment, inference and/or training logic 515 may include, without limitation, hardware logic in which computational resources are dedicated or otherwise exclusively used in conjunction with weight values or other information corresponding to one or more layers of neurons within a neural network. In at least one embodiment, inference and/or training logic 515 illustrated in FIG. 5B may be used in conjunction with an application-specific integrated circuit (ASIC), such as Tensorflow® Processing Unit from Google, an inference processing unit (IPU) from Graphcore™, or a Nervana® (e.g., “Lake Crest”) processor from Intel Corp. In at least one embodiment, inference and/or training logic 515 illustrated in FIG. 5B may be used in conjunction with central processing unit (CPU) hardware, graphics processing unit (GPU) hardware or other hardware, such as field programmable gate arrays (FPGAs). In at least one embodiment, inference and/or training logic 515 includes, without limitation, data storage 501 and data storage 505, which may be used to store weight values and/or other information, including bias values, gradient information, momentum values, and/or other parameter or hyperparameter information. In at least one embodiment illustrated in FIG. 5B, each of data storage 501 and data storage 505 is associated with a dedicated computational resource, such as computational hardware 502 and computational hardware 506, respectively. In at least one embodiment, each of computational hardware 506 comprises one or more ALUs that perform mathematical functions, such as linear algebraic functions, only on information stored in data storage 501 and data storage 505, respectively, result of which is stored in activation storage 520.

In at least one embodiment, each of data storage 501 and 505 and corresponding computational hardware 502 and 506, respectively, correspond to different layers of a neural network, such that resulting activation from one “storage/computational pair 501/502” of data storage 501 and computational hardware 502 is provided as an input to next “storage/computational pair 505/506” of data storage 505 and computational hardware 506, in order to mirror conceptual organization of a neural network. In at least one embodiment, each of storage/computational pairs 501/502 and 505/506 may correspond to more than one neural network layer. In at least one embodiment, additional storage/computation pairs (not shown) subsequent to or in parallel with storage computation pairs 501/502 and 505/506 may be included in inference and/or training logic 515.

### Neural Network Training and Deployment

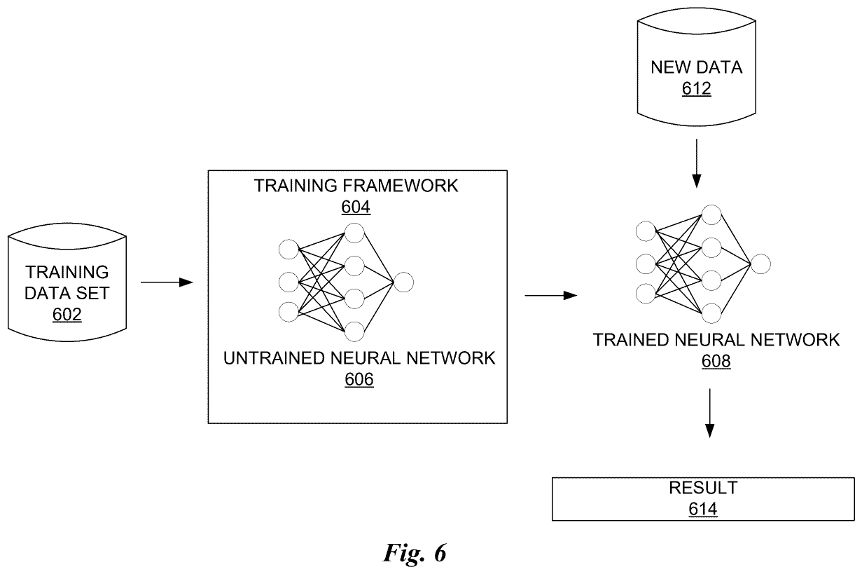

FIG. 6 illustrates another embodiment for training and deployment of a deep neural network. In at least one embodiment, untrained neural network 606 is trained using a training dataset 602. In at least one embodiment, training framework 604 is a PyTorch framework, whereas in other embodiments, training framework 604 is a Tensorflow, Boost, Caffe, Microsoft Cognitive Toolkit/CNTK, MXNet, Chainer, Keras, Deeplearning4j, or other training framework. In at least one embodiment training framework 604 trains an untrained neural network 606 and enables it to be trained using processing resources described herein to generate a trained neural network 608. In at least one embodiment, weights may be chosen randomly or by pre-training using a deep belief network. In at least one embodiment, training may be performed in either a supervised, partially supervised, or unsupervised manner.

In at least one embodiment, untrained neural network 606 is trained using supervised learning, wherein training dataset 602 includes an input paired with a desired output for an input, or where training dataset 602 includes input having known output and the output of the neural network is manually graded. In at least one embodiment, untrained neural network 606 is trained in a supervised manner processes inputs from training dataset 602 and compares resulting outputs against a set of expected or desired outputs. In at least one embodiment, errors are then propagated back through untrained neural network 606. In at least one embodiment, training framework 604 adjusts weights that control untrained neural network 606. In at least one embodiment, training framework 604 includes tools to monitor how well untrained neural network 606 is converging towards a model, such as trained neural network 608, suitable to generating correct answers, such as in result 614, based on known input data, such as new data 612. In at least one embodiment, training framework 604 trains untrained neural network 606 repeatedly while adjust weights to refine an output of untrained neural network 606 using a loss function and adjustment algorithm, such as stochastic gradient descent. In at least one embodiment, training framework 604 trains untrained neural network 606 until untrained neural network 606 achieves a desired accuracy. In at least one embodiment, trained neural network 608 can then be deployed to implement any number of machine learning operations.

In at least one embodiment, untrained neural network 606 is trained using unsupervised learning, wherein untrained neural network 606 attempts to train itself using unlabeled data. In at least one embodiment, unsupervised learning training dataset 602 will include input data without any associated output data or “ground truth” data. In at least one embodiment, untrained neural network 606 can learn groupings within training dataset 602 and can determine how individual inputs are related to untrained dataset 602. In at least one embodiment, unsupervised training can be used to generate a self-organizing map, which is a type of trained neural network 608 capable of performing operations useful in reducing dimensionality of new data 612. In at least one embodiment, unsupervised training can also be used to perform anomaly detection, which allows identification of data points in a new dataset 612 that deviate from normal patterns of new dataset 612.

In at least one embodiment, semi-supervised learning may be used, which is a technique in which in training dataset 602 includes a mix of labeled and unlabeled data. In at least one embodiment, training framework 604 may be used to perform incremental learning, such as through transferred learning techniques. In at least one embodiment, incremental learning enables trained neural network 608 to adapt to new data 612 without forgetting knowledge instilled within network during initial training.

### Data Center

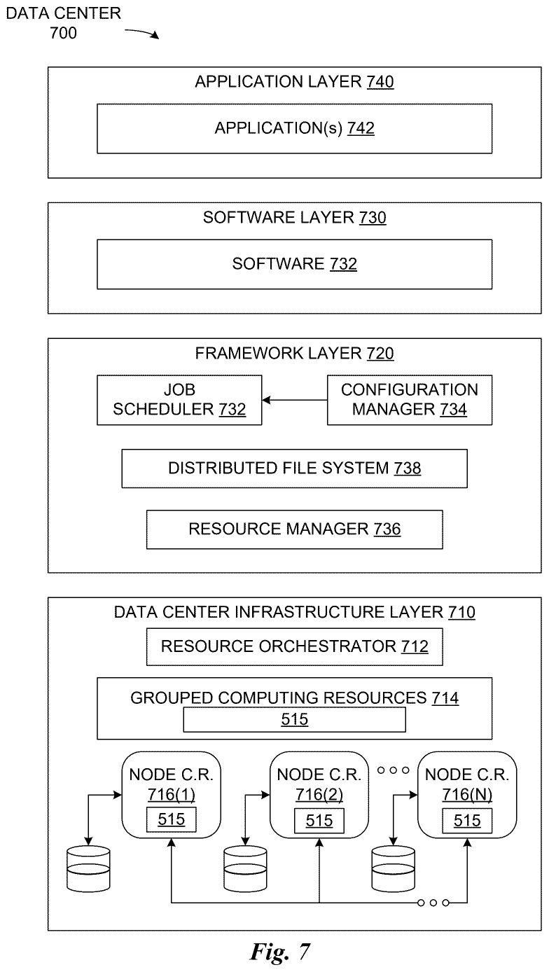

FIG. 7 illustrates an example data center 700, in which at least one embodiment may be used. In at least one embodiment, data center 700 includes a data center infrastructure layer 710, a framework layer 720, a software layer 730 and an application layer 740.

In at least one embodiment, as shown in FIG. 7, data center infrastructure layer 710 may include a resource orchestrator 712, grouped computing resources 714, and node computing resources (“node C.R.s”) 716(1)-716(N), where “N” represents any whole, positive integer. In at least one embodiment, node C.R.s 716(1)-716(N) may include, but are not limited to, any number of central processing units (“CPUs”) or other processors (including accelerators, field programmable gate arrays (FPGAs), graphics processors, etc.), memory devices (e.g., dynamic read-only memory), storage devices (e.g., solid state or disk drives), network input/output (“NW I/O”) devices, network switches, virtual machines (“VMs”), power modules, and cooling modules, etc. In at least one embodiment, one or more node C.R.s from among node C.R.s 716(1)-716(N) may be a server having one or more of above-mentioned computing resources.

In at least one embodiment, grouped computing resources 714 may include separate groupings of node C.R.s housed within one or more racks (not shown), or many racks housed in data centers at various geographical locations (also not shown). Separate groupings of node C.R.s within grouped computing resources 714 may include grouped compute, network, memory or storage resources that may be configured or allocated to support one or more workloads. In at least one embodiment, several node C.R.s including CPUs or processors may grouped within one or more racks to provide compute resources to support one or more workloads. In at least one embodiment, one or more racks may also include any number of power modules, cooling modules, and network switches, in any combination.

In at least one embodiment, resource orchestrator 722 may configure or otherwise control one or more node C.R.s 716(1)-716(N) and/or grouped computing resources 714. In at least one embodiment, resource orchestrator 722 may include a software design infrastructure (“SDI”) management entity for data center 700. In at least one embodiment, resource orchestrator may include hardware, software or some combination thereof.

In at least one embodiment, as shown in FIG. 7, framework layer 720 includes a job scheduler 732, a configuration manager 734, a resource manager 736 and a distributed file system 738. In at least one embodiment, framework layer 720 may include a framework to support software 732 of software layer 730 and/or one or more application(s) 742 of application layer 740. In at least one embodiment, software 732 or application(s) 742 may respectively include web-based service software or applications, such as those provided by Amazon Web Services, Google Cloud and Microsoft Azure. In at least one embodiment, framework layer 720 may be, but is not limited to, a type of free and open-source software web application framework such as Apache Spark™ (hereinafter “Spark”) that may utilize distributed file system 738 for large-scale data processing (e.g., “big data”). In at least one embodiment, job scheduler 732 may include a Spark driver to facilitate scheduling of workloads supported by various layers of data center 700. In at least one embodiment, configuration manager 734 may be capable of configuring different layers such as software layer 730 and framework layer 720 including Spark and distributed file system 738 for supporting large-scale data processing. In at least one embodiment, resource manager 736 may be capable of managing clustered or grouped computing resources mapped to or allocated for support of distributed file system 738 and job scheduler 732. In at least one embodiment, clustered or grouped computing resources may include grouped computing resource 714 at data center infrastructure layer 710. In at least one embodiment, resource manager 736 may coordinate with resource orchestrator 712 to manage these mapped or allocated computing resources.

In at least one embodiment, software 732 included in software layer 730 may include software used by at least portions of node C.R.s 716(1)-716(N), grouped computing resources 714, and/or distributed file system 738 of framework layer 720. one or more types of software may include, but are not limited to, Internet web page search software, e-mail virus scan software, database software, and streaming video content software.

In at least one embodiment, application(s) 742 included in application layer 740 may include one or more types of applications used by at least portions of node C.R.s 716(1)-716(N), grouped computing resources 714, and/or distributed file system 738 of framework layer 720. one or more types of applications may include, but are not limited to, any number of a genomics application, a cognitive compute, and a machine learning application, including training or inferencing software, machine learning framework software (e.g., PyTorch, TensorFlow, Caffe, etc.) or other machine learning applications used in conjunction with one or more embodiments.

In at least one embodiment, any of configuration manager 734, resource manager 736, and resource orchestrator 712 may implement any number and type of self-modifying actions based on any amount and type of data acquired in any technically feasible fashion. In at least one embodiment, self-modifying actions may relieve a data center operator of data center 700 from making possibly bad configuration decisions and possibly avoiding underutilized and/or poor performing portions of a data center.

In at least one embodiment, data center 700 may include tools, services, software or other resources to train one or more machine learning models or predict or infer information using one or more machine learning models according to one or more embodiments described herein. For example, in at least one embodiment, a machine learning model may be trained by calculating weight parameters according to a neural network architecture using software and computing resources described above with respect to data center 700. In at least one embodiment, trained machine learning models corresponding to one or more neural networks may be used to infer or predict information using resources described above with respect to data center 700 by using weight parameters calculated through one or more training techniques described herein.

In at least one embodiment, data center may use CPUs, application-specific integrated circuits (ASICs), GPUs, FPGAs, or other hardware to perform training and/or inferencing using above-described resources. Moreover, one or more software and/or hardware resources described above may be configured as a service to allow users to train or performing inferencing of information, such as image recognition, speech recognition, or other artificial intelligence services.

Inference and/or training logic 515 are used to perform inferencing and/or training operations associated with one or more embodiments. In at least one embodiment, inference and/or training logic 515 may be used in system FIG. 7 for inferencing or predicting operations based, at least in part, on weight parameters calculated using neural network training operations, neural network functions and/or architectures, or neural network use cases described herein.

As described herein, a method, computer readable medium, and system are disclosed to provide protein binder design synthesis. In accordance with FIGS. 1A-4, embodiments may provide a flow-based generative model usable for performing inferencing operations and for providing inferenced data (e.g. a synthetic protein binder design). The flow-based generative model may be stored (partially or wholly) in one or both of data storage 501 and 505 in inference and/or training logic 515 as depicted in FIGS. 5A and 5B. Training and deployment of the flow-based generative model may be performed as depicted in FIG. 6 and described herein. Distribution of the flow-based generative model may be performed using one or more servers in a data center 700 as depicted in FIG. 7 and described herein.

What is claimed is:

1. A method, comprising:
at a device:
training a flow-based generative model to generate a synthetic protein binder design conditioned on a target protein structure; and
outputting the trained flow-based generative model.

2. The method of claim 1, wherein the flow-based generative model is trained to generate a novel, fully atomistic protein binder predicted to interact with the target protein structure.

3. The method of claim 1, wherein the flow-based generative model is pretrained.

4. The method of claim 3, wherein the flow-based generative model is pretrained on monomeric proteins, to learn a partially latent representation of proteins.

5. The method of claim 1, wherein the flow-based generative model is trained on pairs of protein binders and target proteins to learn their structural relationships.

6. The method of claim 5, wherein the flow-based generative model includes a denoiser that is trained using each of the pairs of protein binders and target proteins by:
adding noise to the protein binder,
predicting, by the flow-based generative model, velocities relative to the noise, and
optimizing the flow-based generative model based on the prediction.

7. The method of claim 1, wherein the flow-based generative model is trained to only generate the synthetic protein binder design without generating a full target-binder complex.

8. The method of claim 1, wherein the method further comprises, at the device:
performing reward-based fine-tuning of the trained flow-based generative model by:
calculating a reward based on predefined structural metrics that are favorable for successful synthetic protein binder design, and
updating parameters of the trained flow-based generative model based on the reward.

9. The method of claim 1, wherein the trained flow-based generative model is output to an execution environment for performing inference-time operations.

10. The method of claim 9, wherein the inference-time operations include generating a new synthetic protein binder design conditioned on an input target protein structure.

11. A method, comprising:
at a device:
generating a synthetic protein binder design conditioned on a target protein structure, by a flow-based generative model; and
outputting the synthetic protein binder design.

12. The method of claim 11, wherein the synthetic protein binder design is a novel, fully atomistic protein binder predicted to interact with the target protein structure.

13. The method of claim 11, wherein the flow-based generative model generates only the synthetic protein binder design without generating a full target-binder complex.

14. The method of claim 11, wherein the synthetic protein binder design generated by the flow-based generative model is further conditioned on a target fold class.

15. The method of claim 11, wherein generating the synthetic protein binder design conditioned on the target protein structure, by the flow-based generative model, includes generating a predefined number of synthetic protein binder designs conditioned on the target protein structure, by the flow-based generative model; and
wherein the method further comprises, at the device:
performing an inference-time optimization on the synthetic protein binder designs to select one or more of the synthetic protein binder designs for output.

16. The method of claim 11, wherein the synthetic protein binder design is generated by the flow-based generative model via an inference-time optimization that includes a rollout process.

17. The method of claim 16, wherein the rollout process includes, for each of a plurality of steps of the rollout process and for each of one or more denoising trajectories:
generating a plurality of candidate states for the denoising trajectory,
scoring each of the plurality of candidate states, and
selecting one or more of the plurality of candidate states to update the denoising trajectory, based on the scores.

18. The method of claim 11, wherein the synthetic protein binder design is output to a memory.

19. The method of claim 11, further comprising:
generating a physical protein from the synthetic protein binder design.

20. A system, comprising:
a non-transitory memory storage comprising instructions; and
one or more processors in communication with the memory, wherein the one or more processors execute the instructions to:
generate a synthetic protein binder design conditioned on a target protein structure, by a flow-based generative model; and
output the synthetic protein binder design.

21. The system of claim 20, further comprising:
a subsystem to generate a physical protein from the synthetic protein binder design.

22. A non-transitory computer-readable media storing computer instructions which when executed by one or more processors of a device cause the device to:
generate a synthetic protein binder design conditioned on a target protein structure, by a flow-based generative model; and
output the synthetic protein binder design.

23. The non-transitory computer-readable media of claim 22, wherein the device further causes a physical protein to be generated from the synthetic protein binder design.
